(19) United States
(12) Patent Application Publication
(10) Pub. No.: US 2023/0248623 A1
(43) Pub. Date: Aug. 10, 2023

(54) WATER-IN-OIL EMULSION HAVING A HIGH INTERNAL WATER PHASE CONTENT

(71) Applicant: L'OREAL, Paris (FR)

(72) Inventors: Pauline DUBUISSON, Chevilly La Rue (FR); Nathalie GUILLIER, Chevilly La Rue (FR)

(21) Appl. No.: 18/004,734

(22) PCT Filed: Jul. 8, 2021
(86) PCT No.: PCT/EP2021/069076

§ 371 Date: Jan. 9, 2023

(30) Foreign Application Priority Data
Jul. 10, 2020 (FR) FR2007347

(51) Int. Cl. A61K 8/06; A61K 8/36; A61Q 1/00; A61Q 19/00; A61K 8/92

(52) U.S. Cl. CPC A61K 8/064; A61K 8/361; A61Q 1/00; A61Q 19/00; A61K 8/92; A61K 2800/48

(57) ABSTRACT

The present application relates to a composition in the form of a water-in-oil emulsion having a high internal water phase content and improved stability and improved sensory qualities, comprising: a) a continuous oil phase containing at least two emulsifiers chosen from esters of C8-C30 fatty acid and polyglycerol, and at least one non-silicone oil with a relative density to water at 4° C. of less than 0.9; and b) an aqueous phase dispersed in the oil phase and constituting a content of at least 70 wt % of the total weight of the composition constituting 100%; characterised in that the ratio by weight of the emulsifiers to the at least one oil is less than 1/5.

### TECHNICAL FIELD

The present invention relates to water-in-oil (W/O) emulsions having a high content of aqueous internal phase (hereinafter abbreviated to HIPE for “high internal phase emulsion”). The invention also relates to a specific emulsifying oily composition and to its use as the oily phase of such a W/O emulsion in order to form a stable HIPE.

### CONTEXT OF THE INVENTION

A conventional emulsion is formed of a high content of external phase and of a smaller content of internal phase in which droplets of the internal phase are surrounded by an emulsifier and dispersed in the external phase. In a high internal phase emulsion (HIPE), the external phase forms a small part of the emulsion. The formation of such an emulsion is made possible by a distortion of the droplets in the internal phase which pass from a spherical shape to a polyhedral shape. This distortion can only be achieved by a rigorous selection of specific emulsifying systems. The polyhedral shape of the droplets makes it possible for the internal phase to be present at a level of 99% of the total volume of the emulsion. In comparison, for a standard form of spherical particles, the internal phase can represent at most approximately 74% of the total volume of the emulsion. Theoretically, a HIPE is thus an emulsion in which the volume of the internal phase represents at least 75% of the total volume of the emulsion.

HIPEs are particularly suitable for cosmetic use, in particular as products to be applied to the skin, because they have an appropriate rheology without the use of additional water thickeners which conventional oil-in-water emulsions require. Likewise, conventional water-in-oil emulsions generally have a low viscosity. In order to thicken water-in-oil emulsions, at least one oil thickener is then required in a large amount. Such thickeners comprise in particular waxes, modified clays, such as Bentone Gel®, or also fumed silica. These oil thickeners tend to make the cosmetic product heavy and greasy on application, which can constitute a hindrance to their use.

One of the other advantages of water-in-oil emulsions of HIPE type is that they contain low contents of oil, and are thus much less expensive to produce than conventional water-in-oil emulsions.

However, current HIPEs still do not have sufficient levels of stability, in particular at 45° C. The formulation of stable W/O emulsions with a content of oily phase of less than 30% by weight still remains a challenge. Either the emulsifiers available do not make possible sufficient stability, or the emulsion obtained has a viscosity which is too high. Moreover, during the production of HIPE emulsions, it is often necessary to heat at least certain ingredients and/or aqueous and/or oily phases before or during their mixing, and the mixing of oil and water generally requires high shearing. It would thus be advantageous to be able to produce the emulsion without the need to heat the reactants so that the active ingredients, in particular the temperature-sensitive active principles, can be included directly either in the oily phase or in the aqueous phase before the mixing.

Despite the recognized advantages of W/O emulsions, their use is limited by their sensory profile, which is often considered too heavy, and/or too greasy, indeed even tacky, in particular for conventional W/O emulsions. Conventional inverse emulsions are known to leave a greasy film on the skin, in particular due to the presence of emulsifiers. Silicone oils are often combined with them in order to limit this impact of said emulsifiers. Commercially available HIPEs thus generally contain silicone oils. Such emulsions are described, for example, in the patent documents KR20140070719 and US2003/0064046. Moreover, more and more consumers are asking for natural products, in particular excluding silicone oils.

A search is thus underway to formulate emulsions which do not have the abovementioned disadvantages, in particular comprising a content of surfactant which is as low as possible, and not requiring the use of silicone oils. It is thus an aim of the present invention to provide compositions in the form of W/O emulsions with a high content of internal phase, which are simultaneously stable, non-greasy, easy to formulate and preferably using the most natural ingredients possible.

Another aim of the present invention is to provide compositions comprising an aqueous phase and a fatty phase, the process of production of which is as simple as possible, comprises the fewest possible stages, does not require heating the ingredients, nor high levels of shearing to emulsify the two phases.

The applicant company has shown that, surprisingly, the combination of two specific surfactants and of a selection of oil of specific density made it possible to obtain inverse emulsions with a high content of aqueous phase with sensory qualities unexpected for emulsions of this type, such as softness and freshness, which are stable over time. To obtain these results, no silicone oil or thickener is necessary, so that the W/O emulsions with a high content of aqueous phase of the invention exhibit only a slight impact on the environment.

### SUMMARY OF THE INVENTION

A subject matter of the present invention is thus a composition (A) in the form of a water-in-oil emulsion comprising:

a) an oily composition constituting the continuous oily phase containing:
at least two emulsifiers, implying different, that is to say at least partially different in chemical composition, chosen from esters of C₈-C₃₀ fatty acid and of polyglycerol, and
at least one nonsilicone oil with a density of less than 0.9 (density relative to water at 4° C.);
   and
      b) an aqueous composition constituting the aqueous phase dispersed in said oily phase a) and representing a content of at least 70% by weight, with respect to the total weight of the composition (A) representing 100%;
      characterized in that the ratio by weight of said emulsifiers to said at least one oil is less than 1/5. Preferably, the ratio by weight of said emulsifiers to said at least one oil is between 1/25 and 1/5.

Another subject matter of the present invention is an oily emulsifying composition a) as defined according to the invention, comprising:
at least two emulsifiers chosen from esters of C₈-C₃₀ fatty acid and of polyglycerol, and
at least one nonsilicone oil with a density of less than 0.9 (density relative to water at 4° C.);
   characterized in that the ratio by weight of said emulsifiers to said at least one oil is less than 1/5. Preferably, the ratio of the contents by weight of said emulsifiers to said at least one oil is between 1/25 and 1/5.

Another subject matter of the present invention is the use of said oily emulsifying composition a) in the manufacture of a water-in-oil HIP emulsion (A) with improved stability and/or having the greasy effect reduced, and with a content of aqueous internal phase b) of at least 70% by weight, preferably of at least 74% by weight, with respect to the total weight of the composition (A).

Another subject matter of the present invention is a process for the manufacture of a water-in-oil emulsion (A) according to the invention, in which said aqueous phase b), which is homogeneous or homogenized beforehand if necessary, is directly mixed with said continuous oily phase a) at ambient temperature, in particular within the range from 15 to 25° C., preferably gradually, preferably with stirring according to a stirring speed within the range from 200 to 1000 rpm.

A subject matter of the present invention is in particular a formulation kit comprising:
on the one hand, an oily emulsifying composition a) as defined above, and
on the other hand, an aqueous composition b) as defined above,
   in which said aqueous composition b) and said oily composition a) are packaged separately and intended to be mixed at the time of use in order to form a cosmetic product, preferably in the form of a water-in-oil HIPE emulsion (A) according to the invention, in particular a makeup, makeup-removal, cleansing or care product for the skin, mucous membranes, superficial body growths or hair.

A further subject matter of the present invention is a nontherapeutic method for caring for, making up or removing makeup from keratin materials, in particular skin, including scalp, hair and/or lips, comprising the application, to the keratin materials, of a water-in-oil emulsion (A) as defined above.

### DETAILED DESCRIPTION

Within the meaning of the present invention and unless otherwise indicated:

The expression “at least one” is equivalent to “one or more” and, unless otherwise indicated (such as “more than . . . ”, “less than” or “between X and Y”), the limits of a range of values are included in this range (for example, “in the range from X to Y”).

Within the meaning of the invention, and in the continuation of the description, the contents being generally measured and expressed in % by weight, the term “HIPE” or “water-in-oil (W/O) emulsion having a high content of aqueous internal phase” is understood to mean a W/O emulsion comprising at least 70%, preferably from 70% to 95%, preferably from 75% to 90%, by weight of aqueous internal phase b) with respect to the total weight of the emulsion (A). Preferably, the content of aqueous internal phase b) is less than or equal to 95% by weight, with respect to the total weight of the composition (A). This is because, when the internal phase is greater than 95%, in some cases the emulsion can become too thick and too viscous for cosmetic use. Preferably, the composition in the form of a W/O emulsion according to the invention has a viscosity within the range from 5 to 1000 poises (0.5 to 100 Pa·s), preferably from 10 to 500 poises, preferably from 50 to 100 poises.

The “density” of an oil within the meaning of the invention is the relative density of the oil with respect to pure water at 4° C., that is to say the ratio of the density of the oil to the density of pure water at 4° C., taken as reference for the liquids.

Advantageously, the ratio by weight of the aqueous phase b) to the oily phase a) in the composition in the form of emulsion (A) according to the invention is within the range from 70/30 to 95/5, preferably from 74/26 to 95/5, preferably from 75/25 to 95/5, preferably from 75/25 to 90/10, preferably from 80/20 to 90/10. Thus, the aqueous phase b) advantageously represents from 74% to 95%, preferably from 75% to 95%, preferably from 80% to 95%, indeed even from 80% to 90%, by weight, with respect to the total weight of the composition (A) representing 100%.

The term “stable emulsion” is understood to mean, within the meaning of the invention, an emulsion which, after 2 months of storage at 4° C., 25° C. and 45° C., does not exhibit any macroscopic change in color, odor or viscosity but which on the contrary remains homogeneous and uniform and which does not phase separate (no separation of the aqueous phase and the oily phase) or release oil.

The compositions (A) according to the invention can be cosmetic or dermatological compositions. Preferably, they are cosmetic compositions.

The composition (A) according to the invention contains a physiologically acceptable medium.

In the present invention, the term “physiologically acceptable medium” is understood to mean a nontoxic medium which is compatible with the skin (including the interior of the eyelids), the mucous membranes, the hair or the lips of human beings. A cosmetic composition is a product having a pleasant appearance, smell and feel and intended for topical application.

Oily phase or oily emulsifying composition a)

The oily phase a) is also called “oily emulsifying composition” within the meaning of the invention but can also be called “lipophilic phase” or again “liquid fatty phase”.

The oily phase preferably represents from 5% to 30%, preferably from 5% to 25%, preferably from 10% to 25%, preferably from 10% to 20%, by weight, with respect to the total weight of the composition.

Completely unexpectedly, the stability of the HIPE emulsions (A) of the invention is based mainly on the specific combination, in the oily phase a), of at least two, in particular of two, emulsifiers which are esters of C₈-C₃₀ fatty acid and of polyglycerol, in combination with at least one nonsilicone oil with a density of less than 0.9.

### Emulsifiers Which are Esters of Fatty Acid and of Polyglycerol

The term “ester of C₈-C₃₀ fatty acid and of polyglycerol” within the meaning of the invention is understood to mean an ester of fatty acid (or of fatty acid polymer) and of polyglycerol in which the fatty acid comprises a saturated or unsaturated, linear or branched, C₈-C₃₀ hydrocarbon chain, which is preferably C₁₆-C₂₀, and preferably branched.

According to one embodiment, the fatty acid is in a polymeric form, as is the case, for example, for polyhydroxystearic acid (polymer of 12-hydroxystearic acid).

Advantageously, said C₈-C₃₀ fatty acids having a linear or branched chain in the emulsifier are chosen from: stearic acid, isostearic acid, lauric acid, myristic acid, palmitic acid, palmitoleic acid, arachidic acid, arachidonic acid, behenic acid, lignoceric acid or cerotic acid.

Preferably, the fatty acids in the esters of fatty acid and of polyglycerol used in the present invention comprise a C₁₆-C₂₀ hydrocarbon chain and are independently chosen from stearic acid, isostearic acid, palmitic acid or arachidic acid. Mention made be made, as example of C₁₆-C₂₀ fatty acid polymer, of poly(12-hydroxystearic acid).

Advantageously, the fatty acids of said at least two ester emulsifiers of the composition a) and/or (A) according to the invention are identical (the glycerolated part then being different) or else the fatty acids are different (it being possible for the glycerolated part to be identical or different) and preferably chosen independently from: stearic acid, isostearic acid, poly(l2-hydroxystearic acid) and their mixtures.

The term “polyglycerol” is understood to mean a compound of following formula:

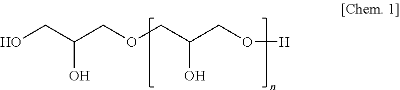

in which the degree of condensation n ranges from 1 to 11, preferably from 2 to 6 and more preferentially still from 3 to 6.

Preferably, the ester of fatty acid and of polyglycerol contains from 2 to 10 mol (or units) of polyols, preferably from 2 to 4 units of polyols, in particular from 2 to 4 glycerol units or a mixture of polyglycerols (glycerol, di-, tri-, tetra-, penta-, oligoglycerols). The terms “mole” and “unit” are used without distinction here.

More preferentially still, the ester of fatty acid and of polyglycerol contains 3 or 4 glycerol units.

According to a specific embodiment, said ester of fatty acid and of polyglycerol is additionally an ester of fatty acid, of dicarboxylic acid having from 2 to 16 carbon atoms, preferably from 8 to 14 carbon atoms, such as azelaic acid, sebacic acid or dodecanedioic acid, and preferably sebacic (C₁₀) acid, and of polyglycerol.

Mention may be made, as examples of esters of fatty acid and of polyglycerol which can be used in the composition of the invention, of esters of isostearic acid and of polyglycerol, such as, for example, polyglycerolated (4 units) isostearate of INCI name: Polyglyceryl-4 Isostearate and sold in particular under the name Isolan G134® by Goldschmidt, polyglycerolated (3 units) diisostearate of INCI name Polyglyceryl-3 Diisostearate and sold in particular under the name Lameform TGI® by Cognis; polyglycerolated (2 units) distearate of INCI name Polyglyceryl-2 Distearate sold, for example, under the name Emalex PGSA® by Nihon Emulsion; polyglycerolated (10 units) monoisostearate sold under the name Nikkol Decaglyn 1-IS by Nihon Surfactant (INCI name: Polyglyceryl-10 Isostearate); Polyglyceryl-4 Diisostearate/Polyhydroxystearate/Sebacate sold under the name Isolan GPS by Goldschmidt; and in particular the mixtures or combinations of at least 2, and preferably only 2, of these esters of fatty acid and of polyglycerol in accordance with the composition a) and/or (A) according to the invention.

Advantageously, in the emulsifiers which are esters of C₈-C₃₀ fatty acid and of polyglycerol used according to the invention, said fatty acid of said emulsifiers preferably comprises a C₁₆-C₂₀ alkyl chain, which is preferably branched, and preferably comprises isostearic acid. Advantageously, said esters of fatty acid and of polyglycerol each independently contain from 2 to 10 glycerol units, preferably from 2 to 4 glycerol units, preferably 3 or 4 glycerol units. Preferably, said esters of fatty acid and of polyglycerol are independently chosen from: esters of isostearic acid and of polyglycerol, preferably chosen from those of INCI name: Polyglyceryl-4 Isostearate, Polyglyceryl-3 Diisostearate or Polyglyceryl-4 Diisostearate/Polyhydroxystearate/Sebacate.

Mention may in particular be made, as preferred esters of fatty acid and of polyglycerol according to the invention, of: polyglycerolated (3 units) diisostearate of INCI name Polyglyceryl-3 Diisostearate, polyglycerolated (4 units) isostearate of INCI name: Polyglyceryl-4 Isostearate, Polyglyceryl-4 Diisostearate/Polyhydroxystearate/Sebacate sold under the name Isolan GPS® by Goldschmidt, and in particular the combinations of at least 2, that is to say 2, 3, indeed even more, but preferably simply 2, of these preferred esters of fatty acid and of polyglycerol, in accordance with the composition a) and/or (A) according to the invention.

Preferably, the composition a) and/or (A) according to the invention comprises only two of these esters of fatty acid and of polyglycerol.

According to a first advantageous embodiment of the invention, one of the two esters of fatty acid and of polyglycerol is polyglycerolated (3 units) diisostearate or Polyglyceryl-3 Diisostearate and the second ester of fatty acid and of polyglycerol is polyglycerolated (4 units) isostearate of INCI name: Polyglyceryl-4 Isostearate.

According to another embodiment, one of the two esters of fatty acid and of polyglycerol according to the invention is an ester of poly(l2-hydroxystearic acid) and of dicarboxylic acids obtained by esterification of a mixture of polyglycerol with (i) a polyhydroxystearic acid, with from 1 to 10, preferably from 2 to 8, more preferentially still from 2 to 5, polyglycerol units (preferably 4 units), (ii) linear or branched aliphatic dicarboxylic acids having from 2 to 16 carbon atoms, preferably from 4 to 14 carbon atoms (preferably sebacic acid), and (iii) saturated or unsaturated, linear or branched, fatty acids having from 6 to 22 carbon atoms, preferably from 16 to 20 carbon atoms (preferably isostearic acid).

Advantageously, the degree of esterification of the polyglycerol mixture is between 20% and 40%, preferably between 40% and 70%.

Such esters of poly(l2-hydroxystearic acid) and of polyglycerol are described in the application US 2005/0031580.

According to a preferred embodiment, one of the two esters of fatty acid and of polyglycerol is an ester of poly(l2-hydroxystearic acid) and of dicarboxylic acids obtained by esterification of a mixture of polyglycerol with (i) a polyhydroxystearic acid, with from 2 to 5 polyglycerol units, (ii) linear or branched aliphatic dicarboxylic acids having from 4 to 14 carbon atoms and (iii) saturated or unsaturated, linear or branched, fatty acids having from 16 to 20 carbon atoms (preferably isostearic acid).

Preferably, the ester of fatty acid and of polyol is an ester of polyhydroxystearic acid and of dicarboxylic acids obtained by esterification of a mixture of polyglycerol with (i) a polyhydroxystearic acid, with from 2 to 5 polyglycerol units (preferably 4 units), (ii) linear or branched aliphatic dicarboxylic acids having from 4 to 14 carbon atoms (preferably sebacic acid) and (iii) saturated or unsaturated, linear or branched, fatty acids having from 16 to 20 carbon atoms (preferably isostearic acid).

Mention may be made, as preferred example of ester of polyhydroxystearic acid and of polyglycerol, of polyglyceryl-4 diisostearate/polyhydroxystearate/sebacate of formula:

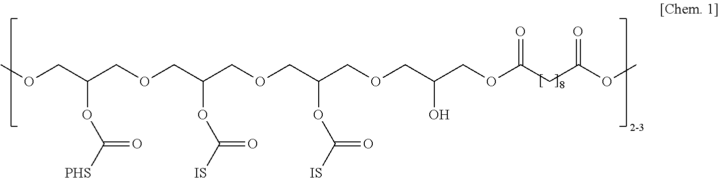

where PHS denotes polyhydroxystearic acid and IS denotes isostearic acid.

Such a compound is prepared according to the application US 2005/0031580 and sold under the name Isolan GPS® by Goldschmidt (Degussa).

More preferably still, polyglyceryl-4 diisostearate/polyhydroxystearate/sebacate sold under the name Isolan GPS® by Goldschmidt is used in the composition a) and/or (A) of the invention.

In this embodiment, the second ester of fatty acid and of polyglycerol is preferably chosen from polyglycerolated (3 units) diisostearate of INCI name Polyglyceryl-3 Diisostearate and polyglycerolated (4 units) isostearate of INCI name: Polyglyceryl-4 Isostearate.

According to a second advantageous embodiment of the invention, one of the two esters of fatty acid and of polyglycerol is polyglyceryl-4 diisostearate/polyhydroxystearate/sebacate and the second ester of fatty acid and of polyglycerol is polyglycerolated (3 mol) diisostearate or Polyglyceryl-3 Diisostearate.

According to a third advantageous embodiment of the invention, one of the two esters of fatty acid and of polyglycerol is polyglyceryl-4 diisostearate/polyhydroxystearate/sebacate and the second ester of fatty acid and of polyglycerol is polyglycerolated (4 units) isostearate of INCI name: Polyglyceryl-4 Isostearate.

Preferably, the total content of said emulsifiers represents, by weight of active material, a content of less than 5%, preferably within the range from 0.1% to 4%, preferably of less than 4%, preferably within the range from 0.2% to 3.5%, preferably from 0.3% to 3%, preferably of less than 3%, preferably within the range from 0.4% to 2.5%, preferably within the range from 0.5% to 2%, preferably of less than 2%, preferably within the range from 0.5% to 1.5% by weight, with respect to the total weight of the composition (A) in emulsion form representing 100%.

Advantageously, in the composition in emulsion form (A) as in the oily emulsifying composition a), the ratio by weight between said emulsifiers, preferably between just the two emulsifiers, is within the range from 1:2 to 2:1, preferably from 1:1.5 to 1.5:1, preferably from 1:1.2 to 1.2:1, preferably from 1:1.1 to 1.1:1, preferably substantially equal to 1. Preferably, the contents by weight of the two emulsifiers, whether with regard to the total weight of oily composition a) or with regard to the total weight of the emulsion (A), are substantially equal.

According to a first preferred embodiment of the invention, the two emulsifiers are respectively Polyglyceryl-4 Isostearate and Polyglyceryl-3 Diisostearate, preferably according to identical contents by weight in the composition a) and/or (A).

According to a second preferred embodiment of the invention, the two emulsifiers are respectively Polyglyceryl-3 Diisostearate and Polyglyceryl-4 Diisostearate/Polyhydroxystearate/Sebacate, preferably according to identical contents by weight in the composition a) and/or (A).

According to a third preferred embodiment of the invention, the two emulsifiers are respectively Polyglyceryl-4 Isostearate and Polyglyceryl-4 Diisostearate/Polyhydroxystearate/Sebacate, preferably according to identical contents by weight in the composition a) and/or (A).

In the composition a) and/or (A) according to the invention, the ratio by weight of said emulsifiers to said at least one nonsilicone oil is less than 1/5. Preferably, the ratio by weight of said emulsifiers to said at least one nonsilicone oil is between 1/25 and 1/5.

Such a ratio of greater than 1/25 in a HIPE emulsion according to the invention guarantees an optimum stability of the HIPE emulsion of greater than 2 months, indeed even of greater than 3 months. Conversely, when said ratio is greater than 1/5, the content of emulsifier is unnecessarily high to the detriment of the sensory properties of the emulsion obtained, such as a shiny and greasy finish on keratin materials.

### The Oils

The term “oils” is understood to mean, within the meaning of the present invention, compounds and/or mixtures of compounds which are insoluble in water, existing in a liquid aspect at a temperature of 25° C.

The oils according to the invention are advantageously chosen from nonsilicone hydrocarbon oils, predominantly comprising carbon and hydrogen atoms, and which can optionally comprise a carbonate or ester group, preferably a single group of this type, within the molecule.

The oils according to the invention are additionally characterized by their density of less than 0.9 (density relative to water at 4° C.).

The oils according to the invention advantageously exhibit one or more (preferably at most 2) fatty chain(s), that is to say long chain(s) comprising at least 8 carbon atoms.

The oils particularly suitable for the compositions according to the invention are chosen from C₈-C₃₀ alkanes and/or dialkyl carbonates of respective C₈-C₃₀ alkyl chains and/or fatty esters of formula R₁COOR₂ where R₁ represents the residue of a fatty acid comprising from 8 to 30 carbon atoms and R₂ represents a linear or branched hydrocarbon chain containing from 6 to 30 carbon atoms. Preferably, the oil is chosen from squalane, linear or branched C₈-C₁₅ alkanes and their mixtures, C₁₅-C₂₈ alkanes and their mixtures, dicaprylyl carbonate, C₁₆-C₂₀ fatty acid esters and the mixtures of these oils, said oil preferably being chosen from those of renewable origin, preferably of vegetable origin.

The selection of oils according to the invention, of low density <0.9 and having long chain(s), as defined according to the invention, generally corresponds to oils of low steric hindrance. This selection of oils with a density <0.9 makes it possible to reproducibly obtain stable HIPE emulsions, unlike oils which do not meet the criterion of density <0.9.

### Alkanes

The oily phase or oily composition of the invention advantageously comprises one or more alkanes with a relative density of less than 0.9.

Alkane is understood to mean any “hydrocarbon oil” formed essentially, indeed even consisting, of carbon and hydrogen atoms and devoid of heteroatoms, such as N, O, Si and P.

Mention may be made, as examples of hydrocarbon oils, of hydrocarbon oils such as squalene, linear or branched hydrocarbons such as paraffin, petrolatum and naphthalene oils, polybutene, isoeicosane, squalane and their mixtures.

Mention may be made, as hydrocarbon oil, of oils having from 8 to 16 carbon atoms and their mixtures, in particular branched C₈-C₁₆ alkanes, such as C₈-C₁₆ isoalkanes (also called isoparaffins), isododecane, isodecane, isohexadecane, and for example the oils sold under the Isopar or Permethyl trade names, and their mixtures.

According to one embodiment, the oils suitable for the invention can be chosen from isododecane, isohexadecane and their mixtures.

Mention may be made, as hydrocarbon oil, of hydrocarbon oils having from 8 to 15 carbon atoms and their mixtures, and in particular linear C₈-C₁₅ alkanes.

Preferably, the alkanes are “linear alkanes” suitable for the invention comprising from 8 to 14 carbon atoms.

Preferably, the “linear alkanes” suitable for the invention comprise from 9 to 14 carbon atoms.

Preferably, the “linear alkanes” suitable for the invention comprise from 10 to 14 carbon atoms.

Preferably, the “linear alkanes” suitable for the invention comprise from 11 to 14 carbon atoms.

A linear alkane suitable for the invention is advantageously of vegetable origin.

Preferably, the linear alkane or the mixture of linear alkanes present in the composition according to the invention comprises at least one ¹⁴C isotope of carbon (carbon-14); in particular, the ¹⁴C isotope can be present in a ¹⁴C/¹²C ratio of greater than or equal to 1×10⁻16, preferably of greater than or equal to 1×10⁻¹⁵, more preferably of greater than or equal to 7.5×10⁻¹⁴ and better still of greater than or equal to 1.5×10⁻¹³. Preferably, the ¹⁴C/¹²C ratio ranges from 6×10⁻¹³ to 1.2×10^{-12.}

The amount of ¹⁴C isotopes in the linear alkane or the mixture of linear alkanes can be determined by methods known to a person skilled in the art, such as the Libby counting method, liquid scintillation spectrometry or also accelerator mass spectrometry.

Such an alkane can be obtained, directly or in several stages, from a vegetable starting material, such as an oil, a butter, a wax, and the like.

Mention may be made, as examples of alkanes suitable for the invention, of the alkanes described in the patent applications of Cognis WO 2007/068371 or WO 2008/155059 (mixtures of different alkanes differing by at least one carbon). These alkanes are obtained from fatty alcohols, themselves obtained from copra oil or palm oil.

Mention may be made, as examples of linear alkanes suitable for the invention, of n-octane (C₈), n-nonane (C₉), n-decane (C₁₀), n-undecane (C₁₁), n-dodecane (C₁₂), n-tridecane (C₁₃), n-tetradecane (C₁₄) and their mixtures. According to a specific embodiment, the linear alkane is chosen from n-nonane, n-undecane, n-dodecane, n-tridecane, n-tetradecane and their mixtures.

According to a preferred embodiment, mention may be made of the mixtures of n-undecane (C₁₁) and of n-tridecane (C₁₃) obtained in examples 1 and 2 of the application WO 2008/155059 of Cognis.

Mention may also be made of n-dodecane (C₁₂) and n-tetradecane (C₁₄) sold by Sasol respectively under the references Parafol 12-97 and Parafol 14-97, and also their mixtures.

It will be possible to use the linear alkane alone.

Alternatively or preferentially, it will be possible to use a mixture of at least two distinct linear alkanes, differing from one another by a carbon number n of at least 1, in particular differing from one another by a carbon number of 1 or of 2.

According to a first embodiment, a mixture of at least two distinct linear alkanes comprising from 10 to 14 carbon atoms and differing from one another by a carbon number of at least 1 is used. Mention may in particular be made, by way of examples, of mixtures of linear C₁/C₁₁, C₁₁/C₁₂ or C₁₂/C₁₃ alkanes.

According to another embodiment, a mixture of at least two distinct linear alkanes comprising from 10 to 14 carbon atoms and differing from one another by a carbon number of at least 2 is used. Mention may in particular be made, by way of examples, of mixtures of linear C₁₀/C₁₂ or C₁₂/C₁₄ alkanes, for an even carbon number n, and of the C₁₁/C₁₃ mixture, for an odd carbon number n.

According to a preferred embodiment, a mixture of at least two distinct linear alkanes comprising from 10 to 14 carbon atoms and differing from one another by a carbon number of at least 2, and in particular a mixture of linear C₁₁/C₁₃ alkanes or a mixture of linear C₁₂/C₁₄ alkanes, is used.

Other mixtures combining more than two linear alkanes according to the invention, such as, for example, a mixture of at least three distinct linear alkanes comprising from 7 to 14 carbon atoms and differing from one another by a carbon number of at least 1, also form part of the invention but mixtures of two linear alkanes according to the invention are preferred (binary mixtures), said two linear alkanes preferably representing more than 95% and better still more than 99% by weight of the total content of linear alkanes in the mixture. According to a specific embodiment of the invention, in a mixture of linear alkanes, the linear alkane having the lowest carbon number is predominant in the mixture.

According to another embodiment of the invention, a mixture of linear alkanes, in which the linear alkane having the highest carbon number is predominant in the mixture, is used.

Mention may in particular be made, as examples of mixtures suitable for the invention, of the following mixtures:

from 50% to 90% by weight, preferably from 55% to 80% by weight, more preferentially from 60% to 75% by weight, of linear Cn alkane with n ranging from 7 to 14,

from 10% to 50% by weight, preferably from 20% to 45% by weight, preferably from 25% to 40% by weight, of linear Cₙ₊ₓ alkane with x greater than or equal to 1, preferably x=1 or x=2, with n+x of between 8 and 14, with respect to the total weight of the alkanes in said mixture.

In particular, said mixture of alkanes according to the invention contains:

less than 2% by weight, preferably less than 1% by weight, of branched hydrocarbons, and/or less than 2% by weight, preferably less than 1% by weight, of aromatic hydrocarbons, and/or less than 2% by weight, preferably less than 1% by weight and preferentially less than 0.1% by weight, of unsaturated hydrocarbons in the mixture.

More particularly, a linear alkane suitable for the invention can be employed in the form of an n-undecane/n-tridecane mixture.

Advantageously, a mixture of linear alkanes comprising from 55% to 80% by weight, preferably from 60% to 75% by weight, of linear C₁₁ alkane (n-undecane) and from 20% to 45% by weight, preferably from 24% to 40% by weight, of linear C₁₃ alkane (n-tridecane), with respect to the total weight of the alkanes in said mixture, is used.

According to a specific embodiment, the mixture of alkanes is an n-undecane/n-tridecane mixture. In particular, such a mixture can be obtained according to example 1 or example 2 of WO 2008/155059.

According to another specific embodiment, the n-dodecane sold under the reference Parafol 12-97 by Sasol is used.

According to another specific embodiment, the n-tetradecane sold under the reference Parafol 14-97 by Sasol is used.

According to yet another embodiment, a mixture of n-dodecane and n-tetradecane is used.

The composition of the invention can comprise from 1% to 30% by weight of alkane(s), which are preferably linear, in particular from 5% to 25% by weight of alkane(s) and more particularly from 5% to 20% by weight of alkane(s), with respect to the total weight of the composition.

According to a specific embodiment, the composition of the invention comprises at least 50% by weight of alkane(s), which are preferably linear, with respect to the total content of oil(s) of the composition. Preferably, a composition of the invention comprises at least 60%, preferably at least 70%, and more preferably still at least 80%, preferably at least 90% or 100%, of linear alkane(s), with respect to the total content of oil(s) of the composition.

### Fatty Carbonate Esters

The oily composition a) and/or (A) of the present invention advantageously comprises one or more fatty carbonate esters (also called “fatty carbonates”) with a relative density of less than 0.9.

Fatty carbonate esters comprise dialkyl carbonates of following formula:

R₁O(C═O)OR₂, where R₁ and R₂ are independently saturated or unsaturated and linear or branched alkyl chains having from 6 to 30 carbon atoms, or having from 6 to 28 carbon atoms, or having from 6 to 25 carbon atoms, or having from 6 to 22 carbon atoms, for example di(C₁₄₋₁₅ alkyl) carbonate, dicaprylyl carbonate, dihexyl carbonate, diethylhexyl carbonate, dipropylheptyl carbonate, dioctyl carbonate and a mixture of these. In particular, dicaprylyl carbonate is preferred. Specifically, use is preferentially made of esters, the fatty chains of which are of vegetable origin, such as, for example, dicaprylyl carbonate (Cetiol® CC).

According to a specific embodiment, the composition a) and/or (A) of the invention comprises at least 50% by weight of fatty carbonate ester(s), with respect to the total content of oil(s) (thus representing 100% here) of the composition a) and/or (A) considered. Preferably, a composition of the invention comprises at least 60%, preferably at least 70%, and more preferably still at least 80%, preferably at least 90%, indeed even 100%, of fatty carbonate ester(s), with respect to the total content (100%) of oil(s) of the composition.

### Fatty Esters

The oily composition a) of the present invention advantageously comprises one or more fatty esters with a relative density of less than 0.9, chosen in particular from esters, in particular of fatty acids, such as the oils of formulae R₁COOR₂ in which R₁ represents the residue of a fatty acid comprising from 8 to 30 carbon atoms and R₂ represents a linear or branched hydrocarbon chain containing from 6 to 30 carbon atoms, such as, for example, isononyl isononanoate, isopropyl myristate, isopropyl palmitate, 2-ethylhexyl palmitate (or octyl palmitate), 2-octyldodecyl stearate, 2-octyldodecyl erucate or isostearyl isostearate, hydroxylated esters, such as isostearyl lactate, octyl hydroxystearate or octyldodecyl hydroxystearate, fatty alcohol heptanoates, octanoates or decanoates, or any other ester provided that its relative density is less than 0.9, and their mixtures.

Preferably, in a composition (A) according to the invention, the total content of oil chosen from alkanes and/or fatty carbonate esters and/or fatty esters, as are defined above, varies from 5% to 30% by weight, with respect to the total weight of the composition (A), preferably from 5% to 25% by weight and better still from 5% to 20% by weight, indeed even from 10% to 20% by weight, with respect to the total weight of the composition.

Preferably, the oily composition a) and thus the composition (A) contains at least one hydrocarbon oil of natural origin, preferably of vegetable origin. Preferably, the oily composition a) and thus the composition (A) comprises solely oils of natural origin and preferably of vegetable origin.

The oils which can be used in the composition of the invention are thus chosen from oils characterized by their density <0.9, preferably their long chain(s). These oils preferably also have a low steric hindrance. Oil of “low steric hindrance”, within the meaning of the invention, is generally understood to mean oils having a linear or branched hydrocarbon fatty chain (of at least 8 carbon atoms), which can optionally include a single ester or carbonate group, but which do not include another group, for example diacids and diesters being excluded, or in particular a group introducing volume trouble liable to reduce the linearity of the oil, such as an aromatic group, or amide group, or amino acid group, or glyceride group, triglycerides and polyol esters generally already being excluded from the definition of the oil with a density <0.9 according to the invention.

In addition, the composition (A) according to the invention contains less than 5%, preferably less than 1%, preferably less than 0.5%, indeed even better still is devoid:
of polymer, in particular synthetic polymer, and/or
of silicone oil, and/or
of gelling agent or thickener, and/or
of fatty substance of synthetic origin, and/or
of oil with a density of greater than 0.9,
of any fatty substance other than an oil as defined according to the invention and in particular of any fatty substance other than an oil chosen from said alkanes and/or said dialkyl carbonates and/or said fatty esters as are defined above.

### Aqueous Phase

The aqueous phase b) generally represents from 70% to 95% by weight, with respect to the total weight of the composition (A). The aqueous phase advantageously represents from 74% to 95%, preferably from 75% to 90%, preferably from 80% to 90%, by weight, with respect to the total weight of the composition (A) representing 100%.

The aqueous phase b) (also called hydrophilic phase) of the composition according to the invention advantageously comprises water, and preferably at least 50%, preferably at least 60%, indeed even at least 65%, or better still at least 70%, by weight of water, with regard to the total weight of the composition (A).

The water used can be sterile demineralized water and/or a floral water, such as rose water, cornflower water, camomile water or lime blossom water, and/or a natural thermal or mineral water, such as, for example: Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Eaux Bonnes water, Rochefort water, Saint Christau water, Les Fumades water, Tercis-les-Bains water and Avène water. The aqueous phase can also comprise reconstituted thermal water, that is to say a water containing trace elements, such as zinc, copper, magnesium, and the like, reconstituting the characteristics of a thermal water.

The aqueous phase b) can also contain hydrophilic adjuvants, among which are polyols, such as glycerol; propanediol; glycols, such as pentylene glycol, propylene glycol, butylene glycol, isoprene glycol and polyethylene glycols, such as PEG-8; sorbitol; sugars, such as glucose, fructose, maltose, lactose or sucrose; and their mixtures.

The polyol of the aqueous phase b) is miscible with water at ambient temperature (25° C.) and can in particular be chosen from polyols having in particular from 2 to 20 carbon atoms, preferably having from 2 to 10 carbon atoms and preferentially having from 2 to 6 carbon atoms, such as glycerol, propylene glycol, butylene glycol, pentylene glycol, hexylene glycol, dipropylene glycol or diethylene glycol;

or also optionally glycol ethers (having in particular from 3 to 16 carbon atoms), such as mono, di- or tripropylene glycol (C₁-C₄)alkyl ethers, mono, di- or triethylene glycol (C₁-C₄)alkyl ethers; and their mixtures.

The polyol miscible with water at ambient temperature can be in the composition in a content ranging from 0.5% to 20% by weight, with respect to the total weight of the composition, and preferably ranging from 3% to 15% by weight, with respect to the total weight of the composition (A).

The aqueous composition according to the invention can additionally comprise a monoalcohol having from 2 to 6 carbon atoms, such as ethanol or isopropanol, in particular in a content ranging from 0.01% to 10% by weight, with respect to the total weight of the invention, and preferably ranging from 1% to 7% by weight, with respect to the total weight of the composition (A).

According to a specific embodiment of the composition (A) of the invention, the aqueous phase b) additionally comprises from 0.5% to 20%, preferably from 3% to 15%, preferably from 5% to 10%, by weight of at least one polyol, preferably chosen from glycerol and/or C₃-C₆ diols, and their mixtures.

The aqueous phase b) also includes any other hydrophilic ingredient, including chosen from the adjuvants described below. The aqueous phase corresponds in particular to the ingredients of the composition (A) which do not appear in the oily phase a), that is to say contains the hydrophilic ingredients, in particular the ingredients other than those of the oily phase a), that is to say other than the oils, the emulsifiers, and optional other lipophilic ingredients.

Advantageously, the composition (A) of the invention additionally comprises at least one cosmetic adjuvant chosen from cosmetic or dermatological active principles, preservatives, antioxidants, fragrances, fillers, pigments, UV screening agents, odor absorbers and coloring materials.

The ingredients and/or active principles will be present in the composition (A) in contents ranging from 0.01% to 20% by weight, preferably from 0.05% to 10%, preferably from 1% to 5% and more preferentially still from 0.1% to 1% by weight, with respect to the total weight of the composition.

These ingredients and/or active principles and also their concentrations must be such that they do not modify the property desired for the composition (A) in the HIPE emulsion form of the invention.

Preferably, ingredients and/or active principles of natural origin are used.

Mention may be made, as fillers which can be used in the composition (A) of the invention, for example, of powders of natural organic materials, such as corn, wheat or rice starches; or also materials of inorganic natural origin, such as silica, talc, clays, such as kaolin, montmorillonite, saponites, laponites and illites.

The amount of fillers is preferably less than or equal to 6% of the total weight of the composition (A) and better still less than or equal to 5% of the total weight of the composition (A). When they are present, these fillers can be in amounts ranging, for example, from 0.05% to 6% by weight and preferably from 0.1% to 5%, preferably from 0.1% to 3%, by weight, with respect to the total weight of the composition (A).

Advantageously, the composition (A) according to the invention comprises at least one active principle chosen from moisturizing agents; agents for combating free radicals; keratolytic and/or desquamating agents; vitamins; antielastase and anticollagenase agents; trace elements; algal and/or plankton extracts; enzymes and coenzymes; flavonoids and/or isoflavonoids; ceramides; antiglycation agents; NO-synthase inhibitors; agents which stimulate the synthesis of dermal or epidermal macromolecules and/or which prevent their degradation; agents which stimulate the proliferation of fibroblasts or keratinocytes and/or the differentiation of keratinocytes; tightening agents; agents for combating pollution and/or free radicals; and dermorelaxing or dermo-decontracting agents; and their mixtures.

The UV screening agents can be organic or inorganic.

Mention may be made, as examples of organic screening agents which are active in the UVA and/or UVB, which can be added to the composition of the invention, for example, of anthranilates; cinnamic derivatives; dibenzoylmethane derivatives; salicylic derivatives, camphor derivatives; triazine derivatives, such as those described in the patent applications U.S. Pat. No. 4,367,390, EP 863 145, EP 517 104, EP 570 838, EP 796 851, EP 775 698, EP 878 469 and EP 933 376; benzophenone derivatives; β□β′-diphenylacrylate derivatives, benzotriazole derivatives, benzimidazole derivatives; imadazolines; bis-benzoazolyl derivatives, such as described in the patents EP 669 323 and U.S. Pat. No. 2,463,264; p-aminobenzoic acid (PABA) derivatives; methylene bis-(hydroxyphenyl benzotriazole) derivatives, such as described in the applications U.S. Pat. Nos. 5,237,071, 5,166,355, GB 2 303 549, DE19726184 and EP 893 119; screening polymers and screening silicones, such as those described in particular in the application WO93/04665; dimers derived from α-alkylstyrene, such as those described in the patent application DE19855649.

Mention may more particularly be made of the following UV screening agents, designated below under their INCI name:

i) para-Aminobenzoic acid derivatives: PABA, Ethyl PABA, Ethyl Dihydroxypropyl PABA, Ethylhexyl Dimethyl PABA, sold in particular under the name Escalol 507 by ISP, Glyceryl PABA, PEG-25 PABA, sold under the name Uvinul P25 by BASF.

ii) Salicylic derivatives:

Homosalate, sold under the name Eusolex HMS by Rona/EM Industries, Ethylhexyl Salicylate, sold under the name Neo Heliopan OS by Haarmann and Reimer, Dipropylene Glycol Salicylate, sold under the name Dipsal by Scher, TEA Salicylate, sold under the name Neo Heliopan TS by Haarmann and Reimer.

iii) Dibenzoylmethane derivatives:

Butyl Methoxydibenzoylmethane, sold in particular under the trade name Parsol 1789 by Hoffmann-La Roche, Isopropyl Dibenzoylmethane.

iv) Cinnamic derivatives:

Ethylhexyl Methoxycinnamate, sold in particular under the trade name Parsol MCX by Hoffmann-La Roche, Isopropyl Methoxycinnamate, Isoamyl Methoxycinnamate, sold under the trade name Neo Heliopan E 1000 by Haarmann and Reimer, Cinoxate, DEA Methoxycinnamate, Diisopropyl Methylcinnamate, Glyceryl Ethylhexanoate Dimethoxycinnamate.

v) β,β′-Diphenylacrylate derivatives:

Octocrylene, sold in particular under the trade name Uvinul N539 by BASF, Etocrylene, sold in particular under the trade name Uvinul N35 by BASF.

vi) Benzophenone derivatives:

Benzophenone-1, sold under the trade name Uvinul 400 by BASF, Benzophenone-2, sold under the trade name Uvinul D50 by BASF, Benzophenone-3 or Oxybenzone, sold under the trade name Uvinul M40 by BASF, Benzophenone-4, sold under the trade name Uvinul MS40 by BASF, Benzophenone-5, Benzophenone-6, sold under the trade name Helisorb 11 by Norquay, Benzophenone-8, sold under the trade name Spectra-Sorb UV-24 by American Cyanamid, Benzophenone-9, sold under the trade name Uvinul DS-49 by BASF, Benzophenone-12.

vii) Benzylidene camphor derivatives:

3-Benzylidene Camphor, sold under the name Mexoryl SD by Chimex, 4-Methylbenzylidene Camphor, sold under the name Eusolex 6300 by Merck, Benzylidene Camphor Sulfonic Acid, manufactured under the name Mexoryl SL by Chimex, Camphor Benzalkonium Methosulfate, manufactured under the name Mexoryl SO by Chimex, Terephthalylidene Dicamphor Sulfonic Acid, manufactured under the name Mexoryl SX by Chimex, Polyacrylamidomethyl Benzylidene Camphor, manufactured under the name Mexoryl SW by Chimex.

viii) Phenylbenzimidazole derivatives:

Phenylbenzimidazole Sulfonic Acid, sold in particular under the trade name Eusolex 232 by Merck, Benzimidazilate, sold under the trade name Neo Heliopan AP by Haarmann and Reimer.

ix) Triazine derivatives:

Anisotriazine, sold under the trade name Tinosorb S by Ciba Geigy, Ethylhexyl Triazone, sold in particular under the trade name Uvinul T1250 by BASF, Diethylhexyl Butamido Triazone, sold under the trade name Uvasorb HEB by Sigma 3V.

x) Phenylbenzotriazole derivatives:

Drometrizole Trisiloxane, sold under the name Silatrizole by Rhodia Chimie, Methylene Bis-Benzotriazolyl Tetramethylbutylphenol, sold in solid form under the trade name Mixxim BB/100 by Fairmount Chemical or in micronized form in aqueous dispersion under the trade name Tinosorb M by Ciba Specialty Chemicals.

xi) Anthranilic derivatives:

Menthyl Anthranilate, sold under the trade name Neo Heliopan MA by Haarmann and Reimer.

xii) Imidazoline derivatives:

Ethylhexyl Dimethoxybenzylidene Dioxoimidazoline Propionate.

xiii) Benzalmalonate derivatives:

Polyorganosiloxane having benzalmalonate functions, sold under the trade name Parsol SLX by Hoffmann-La Roche,

and the mixtures of these screening agents.

The organic UV screening agents which are more particularly preferred are chosen from the following compounds:

Ethylhexyl Salicylate, Butyl Methoxydibenzoylmethane, Ethylhexyl Methoxycinnamate, Octocrylene, Phenylbenzimidazole Sulfonic Acid, Terephthalylidene Dicamphor Sulfonic Acid, Benzophenone-3, Benzophenone-4, Benzophenone-5, 4-Methylbenzylidene Camphor, Benzimidazilate, Anisotriazine, Ethylhexyl Triazone, Diethyl hexyl Butamido Triazone, Methylene Bis-Benzotriazolyl Tetramethylbutylphenol, Drometrizole Trisiloxane and their mixtures.

The total amount of organic UV screening agents in the composition (A) according to the invention is advantageously within the range from 0.1% to 20% by weight, with respect to the total weight of the composition, and preferably from 0.2% to 15% by weight, with respect to the total weight of the composition.

The composition (A) of the invention can contain inorganic screening agents, such as, for example, pigments and nanopigments of metal oxides, which are coated or uncoated, in particular titanium, iron, zirconium, zinc or cerium oxides, and their mixtures, it being possible for these oxides to be in the form of micro- or nanoparticles (nanopigments), which are optionally coated.

Preferably, titanium dioxide is used.

The UV screening agents can be present in an amount of active material ranging from 0.01% to 20% by weight of active material, preferably from 0.1% to 15% by weight and better still from 0.2% to 10% by weight, with respect to the total weight of the composition (A).

Preferably, the composition (A) according to the invention comprises at least one cosmetic active principle chosen from urea and its derivatives; pro-xylane; ceramides; amino acids, vitamins and provitamins, antioxidant compounds, such as baicalin, polydatin or mangiferin, agents for modulating the complexion of the skin, such as tyrosinase inhibitors, retinol and its derivatives.

Advantageously, the composition (A) of the invention can additionally comprise from 0.2% to 2%, preferably from 0.5% to 2%, preferably from 0.7% to 2%, of at least one salt, such as a sodium or magnesium salt, preferably chosen from magnesium sulfate or sodium chloride, and their mixtures. These salts make it possible to further improve the stability of the emulsion, in particular its microscopic stability, especially by limiting Ostwald ripening.

Advantageously, the composition (A) of the invention can additionally comprise from 0.1% to 1% by weight, with respect to the total weight of the composition, of at least one agent having an effect of protecting the formula against microbes or a preserving effect, in particular those chosen from: salicylic acid, glutamic acid, N,N-diacetic acid, tetrasodium salt, pentylene glycol, glyceryl caprylate, vanillin and their mixtures. These ingredients are very particularly preferred because they have no impact on the stability of the emulsion, while effectively protecting and preserving the composition.

### Viscosity of the HIPE Emulsions According to the Invention

The composition (A) of the invention is, for example, provided in the form of a fluid or compact cream. The composition (A) of the invention exhibits, at ambient temperature (25° C.), a viscosity within the range from 70 to 100 poises, preferably within the range from 75 to 90 poises. In the present description of the invention, and in particular in the examples, the viscosity is measured with a Rheomat 180 at 25° C., with spindles suited to the viscosity, in particular spindle 3 or spindle 4 (35 UDM4).

The composition (A) according to the invention finds its application in a large number of cosmetic treatments for the skin, including the scalp, the hair, the nails and/or the mucous membranes, in particular for the care, the cleansing and/or the making up and/or the sun protection of the skin and/or mucous membranes.

Consequently, a subject matter of the present invention is the cosmetic use of the composition (A) as defined above for the treatment of, the protection of, the care of, the removal of makeup from and/or the cleansing of keratin materials, in particular the skin, lips and/or hair, and/or for making up the skin and/or lips.

Another subject matter of the present invention is a nontherapeutic cosmetic method for caring for, making up or removing makeup from or cleansing keratin materials, in particular the skin, including the scalp, hair and/or lips, comprising the application, to the skin, hair and/or lips, of a composition (A) according to the invention as defined above.

The examples below of compositions according to the invention are given by way of illustration and without a limiting nature. The amounts are given therein as % by weight of starting material (of active material), unless otherwise mentioned. The names of the compounds are shown as chemical names or as INCI names.

### EXAMPLE

### Method of Preparation of All the Compositions Tested Below

The oily phase a) is prepared by mixing the oil(s) and the emulsifier(s) at ambient temperature, with mechanical stirring (200 rpm).
The aqueous phase b) is prepared by mixing the water, the optional preservatives, the optional salts (magnesium salt, for example) and the optional polyols (such as glycerol), at ambient temperature, with stirring (200 rpm).
The homogeneous aqueous phase b) is added to the oily phase a) while gradually increasing the stirring up to 1000 rpm.
The stirring is halted.

### Study of Each Emulsifier Used Alone

Each emulsifier was tested in a 3% simplex emulsion, in 27% of oil, in order to emulsify 70% of water (aqueous phase). Three oils were tested: squalane, dicaprylyl carbonate and isopropyl palmitate.

The stability of the emulsions was evaluated after storage at ambient temperature (25° C.) for 72 h. The results of the stabilities of the simplex emulsions are presented in the following Table 1.

TABLE 1

| | | | At 3% in | |
|---|---|---|---|---|
| | | | dicaprylyl | |
| | | | carbonate | At 3% in |
| | | At 3% in | (Cetiol ® | isopropyl |
| TEST | INCI NAME | squalane | CC) | palmitate |
| CP 1 | GLYCERYL ISOSTEARATE | coalescence | coalescence | coalescence |
| CP 2 | GLYCERYL STEARATE | coalescence | coalescence | coalescence |
| CP 3 | GLYCERYL STEARATE | coalescence | coalescence | coalescence |
| CP 4 | SORBITAN STEARATE | sedimentation | sedimentation | sedimentation |
| CP 5 | POLYGLYCERYL-3 | stable | stable | stable |
| | DIISOSTEARATE | | | |
| CP 6 | GLYCERYL STEARATE | coalescence | coalescence | coalescence |
| CP 7 | POLYGLYCERYL-4 | stable | stable | stable |
| | ISOSTEARATE | | | |
| CP 8 | SORBITAN TRIOLEATE | coalescence | coalescence | coalescence |
| CP 9 | POLYGLYCERYL-4 | stable | stable | stable |
| | DIISOSTEARATE/ | | | |
| | POLYHYDROXYSTEARATE/ | | | |
| | SEBACATE | | | |

The emulsifiers tested make it possible to produce inverse emulsions with noteworthy distinctions between the emulsifiers, whatever the three oils:
with glyceryl stearates, glyceryl isostearate or sorbitan trioleate, the emulsions exhibit coalescence after 72 h;
with sorbitan stearate, the emulsions show sedimentation after 72 h;
with polyglyceryl-3 diisostearate, polyglyceryl-4 isostearate or polyglyceryl-4 diisostearate/polyhydroxystearate/sebacate, the emulsions remain stable for 72 h. However, these same emulsions are no longer stable at 2 months.

### Study of the Emulsifiers Used in Combination in Pairs

Each emulsifier was tested at 2% in combination with each of the other eight emulsifiers, also at 2%, in 26% of oil in order to emulsify 70% of water (aqueous phase).

The stability of the emulsions was evaluated after storage at ambient temperature (25° C.) for 72 h.

With the various glyceryl (iso)stearates, the emulsions exhibit coalescence after 72 h.

With sorbitan stearate, polyglyceryl-3 diisostearate, polyglyceryl-4 isostearate or polyglyceryl-4 diisostearate/polyhydroxystearate/sebacate, the emulsions exhibit stability or slight sedimentation at 72 h.

These four emulsifiers giving stable emulsions as a mixture are stabilized for two months.

The formulae and the results of the emulsions produced in squalane are summarized in the following table 2:

TABLE 2

| INCI | Cp10 | Cp11 | Cp12 | Ex13 | Ex14 | Ex15 |
|---|---|---|---|---|---|---|
| Sorbitan Stearate | 2 | 2 | 2 | | | |
| Polyglyceryl-3 Diisostearate | 2 | | | 2 | 2 | |
| Polyglyceryl-4 Isostearate | | 2 | | 2 | | 2 |
| Polyglyceryl-4 Diisostearate/ | | | 2 | | 2 | 2 |
| Polyhydroxystearate/Sebacate | | | | | | |
| Squalane | 26 | 26 | 26 | 26 | 26 | 26 |
| Salicylic Acid | 0.2 | 0.2 | 0.2 | 0.2 | 0.2 | 0.2 |
| Tetrasodium Glutamate Diacetate | 0.2 | 0.2 | 0.2 | 0.2 | 0.2 | 0.2 |
| Propanediol | 5 | 5 | 5 | 5 | 5 | 5 |
| Water | 64.6 | 64.6 | 64.6 | 64.6 | 64.6 | 64.6 |
| Macroscopic stability 2 months | Coalescence | Coalescence | Coalescence | Stable | Stable | Stable |
| ambient temperature and 45° C. | | | | | | |

The same results are observed with the emulsions produced in dicaprylyl carbonate or isopropyl palmitate.

The combinations of emulsifiers including sorbitan stearate do not give stable emulsions at 2 months (coalescence).

In the case of the combinations of two esters of fatty acid and of polyglycerol, the emulsions are macroscopically stable at 2 months at ambient temperature and at 45° C. At the microscopic scale, the droplets are coarser after two months of storage.

These simplex emulsions based on emulsifiers used in combination demonstrate the advantage of selecting a combination of two emulsifiers chosen from esters of branched C₁₆-C₂₀ fatty acid and of polyglycerol, in particular from the following three emulsifiers: polyglyceryl-3 diisostearate, polyglyceryl-4 isostearate and polyglyceryl-4 diisostearate/polyhydroxystearate/sebacate.

### Influence of Salts on the Microscopic Stability

TABLE 3

| INCI US | Ex 16 | Ex 17 | Ex18 | Ex 19 |
|---|---|---|---|---|
| Polyglyceryl-3 | 2 | 2 | 2 | 2 |
| Diisostearate | | | | |
| Polyglyceryl-4 | 2 | 2 | 2 | 2 |
| Isostearate | | | | |
| Squalane | 26 | 26 | 26 | 26 |
| Salicylic Acid | 0.2 | 0.2 | 0.2 | 0.2 |
| Tetrasodium | 0.2 | 0.2 | 0.2 | 0.2 |
| Glutamate | | | | |
| Diacetate | | | | |
| Propanediol | 5 | 5 | 5 | 5 |
| Magnesium | 0.7 | | 2 | |
| Sulfate | | | | |
| Sodium Chloride | | 2 | | |
| Water | 63.9 | 62.6 | 62.6 | 64.6 |
| Microscopic | Stable | Stable | Stable | Ostwald |
| stability 2 | | | | ripening |
| months ambient | | | | |
| temperature and | | | | |
| 45° C. | | | | |

The addition of sodium or magnesium salt to the compositions Ex16 to Ex18 made it possible to further improve the stability of the emulsions, in particular at the microscopic level.

For the salt-free composition of Ex19, on the contrary Ostwald ripening (or molecular diffusion) is noted. However, the Ostwald ripening phenomenon does not cause destabilization of the system of the HIP emulsion.

### Role of the Oils in the Feasibility of a HIPE Emulsion

Different oils (12 in all) were tested with the different combinations of emulsifiers based on the same formula architecture:

The oily phase is composed of 4% of the combination of two emulsifiers chosen from Polyglyceryl-3 Diisostearate, Polyglyceryl-4 Isostearate and Polyglyceryl-4 Diisostearate/Polyhydroxystearate/Sebacate and of 26% of oil, with regard to the total weight of the composition representing 100% (W/O emulsion).

The aqueous phase is composed of 0.2% Salicylic Acid, 0.2% Tetrasodium Glutamate Diacetate, 5% Propanediol, 2% Magnesium Sulfate and 62.6% water, by weight, with regard to the total weight of the composition representing 100% (W/O emulsion).

### Combination 1: Polyglyceryl-3 Diisostearate/Polyglyceryl-4 Isostearate

TABLE 4

| | Cp20 | Cp21 | Cp22 | Cp23 | Ex24 | Ex25 | Ex26 |
|---|---|---|---|---|---|---|---|
| Caprylic/Capric | 26 | | | | | | |
| Triglyceride | | | | | | | |
| Glycine Soja Oil | | 26 | | | | | |
| Butyrospermum | | | 26 | | | | |
| Parkii Butter | | | | | | | |
| Isopropyl Lauroyl | | | | 26 | | | |
| Sarcosinate | | | | | | | |
| Dicaprylyl Carbonate | | | | | 26 | | |
| Undecane (and) | | | | | | 26 | |
| Tridecane | | | | | | | |
| Squalane | | | | | | | 26 |
| Stability at 2 | No | No | No | No | Emulsion | Emulsion | Emulsion |
| months ambient | emulsion | emulsion | emulsion | emulsion | stable 2 | stable 2 | stable 2 |
| temperature and | | | | | months | months | months |
| 45° C. | | | | | | | |

### Combination 2: Polvglyceryl-3 Diisostearate/Polyglyceryl-4 Diisostearate/Polyhydroxystearate/Sebacate

TABLE 5

| | Cp27 | Cp28 | Cp29 | Cp30 | Ex31 | Ex32 | Ex33 |
|---|---|---|---|---|---|---|---|
| Caprylic/Capric | 26 | | | | | | |
| Triglyceride | | | | | | | |
| Glycine Soja Oil | | 26 | | | | | |
| Butyrospermum | | | 26 | | | | |
| Parkii Butter | | | | | | | |
| Isopropyl Lauroyl | | | | 26 | | | |
| Sarcosinate | | | | | | | |
| Dicaprylyl Carbonate | | | | | 26 | | |
| Undecane (and) | | | | | | 26 | |
| Tridecane | | | | | | | |
| Squalane | | | | | | | 26 |
| Stability at 2 | Coalescence | Coalescence | Coalescence | Coalescence | Emulsion | Emulsion | Emulsion |
| months ambient | after 10 days | after 10 days | after 10 days | after 10 days | stable 2 | stable 2 | stable 2 |
| temperature and | | | | | months | months | months |
| 45° C. | | | | | | | |

### Combination 3 (Polyglyceryl-4 Isostearate/Polyglyceryl-4 Isostearate)

TABLE 6

| | Cp34 | Cp35 | Cp36 | Cp37 | Ex38 | Ex39 | Ex40 |
|---|---|---|---|---|---|---|---|
| Caprylic/Capric | 26 | | | | | | |
| Triglyceride | | | | | | | |
| Glycine Soja Oil | | 26 | | | | | |
| Butyrospermum | | | 26 | | | | |
| Parkii Butter | | | | | | | |
| Isopropyl Lauroyl | | | | 26 | | | |
| Sarcosinate | | | | | | | |
| Dicaprylyl Carbonate | | | | | 26 | | |
| Undecane (and) | | | | | | 26 | |
| Tridecane | | | | | | | |
| Squalane | | | | | | | 26 |
| Stability at 2 | No | No | No | No | Emulsion | Emulsion | Emulsion |
| months ambient | emulsion | emulsion | emulsion | emulsion | stable 2 | stable 2 | stable 2 |
| temperature and | | | | | months | months | months |
| 45° C. | | | | | | | |

The selection of the oil plays a role in the stability of the HIP emulsions.

The oils which did not make it possible to produce stable emulsions are: Caprylic/Capric Triglyceride, Glycine Soja Oil, Butyrospermum Parkii Butter and Isopropyl Lauroyl Sarcosinate.

The oils which did make it possible to produce the stable emulsions are: Dicaprylyl Carbonate, Undecane (and) Tridecane, and Squalane.

Other oils tested under the same conditions also with the three abovementioned combinations of emulsifiers (even if not mentioned in tables 4 to 6) also made it possible to produce stable emulsions, namely: Isopropyl Myristate, a mixture of esters of caprylic/capric acids and of C₁₂-C₁₈ fatty alcohols (Coco-Caprylate/Caprate), dodecane of natural origin, and hydrogenated isoparaffin (6-8 mol of isobutylene) (Hydrogenated Polyisobutene). On the contrary, Diisopropyl Sebacate, also tested under the same conditions, did not make it possible to produce a stable emulsion.

### Feasibility of a HIP Emulsion (HIPE) as a Function of the Oil Tested and of its Density

TABLE 6

| | | Feasibility |
|---|---|---|
| Oil (INCI name or common name) | Density | of a HIPE |
| Butyrospermum Parkii Butter | 0.92 | No |
| Diisopropyl Sebacate | 0.93 | No |
| Isopropyl Lauroyl Sarcosinate | 0.92 | No |
| Caprylic/Capric Triglyceride | 0.94 | No |
| Glycine Soja Oil | 0.92 | No |
| Isopropyl Palmitate | 0.85 | Yes |
| Isopropyl Myristate | 0.85 | Yes |
| Mixture of Esters of Caprylic/Capric Acids | 0.85 | Yes |
| and of C₁₂-C₁₈ Fatty Alcohols | | |
| Dicaprylyl Carbonate | 0.89 | Yes |
| Hydrogenated Polyisobutene | 0.82 | Yes |
| Squalane | 0.81 | Yes |
| Undecane (and) Tridecane | 0.74 | Yes |
| Dodecane of Vegetable Origin | 0.75 | Yes |

According to table 6, the density parameter proves to be determining regarding the choice of the oil making possible the production of a HIPE according to the invention. Oils, the density of which is greater than 0.9, did not make it possible to obtain stable HIP emulsions. On the contrary, the oils according to the invention, with a density of less than 0.9, all made it possible, under the same formulation conditions, to obtain emulsions stable for at least 2 months at 4° C., 25° C. and 45° C.

### Influence of the Content of the Emulsifiers on the Sensory Properties

The sensory factors evaluated are the slippery/nonslippery and fresh/nonfresh effect on application and the shiny/nonshiny and greasy/nongreasy skin finish after application.

Comment: 10% of glycerol was added as replacement for water, according to the following formulae compared in table 7.

TABLE 7

| INCI | Cp41 | Ex42 | Cp43 | Ex44 | Cp45 | Ex46 |
|---|---|---|---|---|---|---|
| Polyglyceryl-3 Diisostearate | 2.0 | 0.5 | 2.0 | 0.5 | | |
| Polyglyceryl-4 Isostearate | 2.0 | 0.5 | | | 2.0 | 0.5 |
| Polyglyceryl-4 Diisostearate/ | | | 2.0 | 0.5 | 2.0 | 0.5 |
| Polyhydroxystearate/Sebacate | | | | | | |
| Squalane | 19.0 | 19.0 | 19.0 | 19.0 | 19.0 | 19.0 |
| Salicylic acid | 0.2 | 0.2 | 0.2 | 0.2 | 0.2 | 0.2 |
| Tetrasodium Glutamate Diacetate | 0.1 | 0.1 | 0.1 | 0.1 | 0.1 | 0.1 |
| Magnesium Sulfate | 0.7 | 0.7 | 0.7 | 0.7 | 0.7 | 0.7 |
| Propanediol | 5.0 | 5.0 | 5.0 | 5.0 | 5.0 | 5.0 |
| Glycerol | 10.0 | 10.0 | 10.0 | 10.0 | 10.0 | 10.0 |
| Water | 61.0 | 64.0 | 61.0 | 64.0 | 61.0 | 64.0 |
| Sensory evaluation: | Not very | Slippery, | Not very | Slippery, | Not very | Slippery, |
| | fresh; | fresh, skin | fresh; | fresh, skin | fresh; | fresh, skin |
| | skin finish | finish | skin finish | finish | skin finish | finish |
| | shiny and | nourished, | shiny and | nourished, | shiny and | nourished, |
| | greasy | nonshiny, | greasy | nonshiny, | greasy | nonshiny, |
| | | not very | | slightly | | slightly |
| | | greasy | | greasy | | greasy |

It is seen, from table 7, that all the formulae are stable at 4° C., at ambient temperature (25° C.) and at 45° C., for at least 2 months. The sensory properties of the formulae were compared by a panel of trained people, and assessed:
on application: on the fresh/not very fresh/not fresh effect and slippery/nonslippery criteria and
for the skin finish: shiny/nonshiny and greasy/nongreasy; with the following results:

Sensory nature of the formulae with 4% of emulsifiers: The application is slippery, not very fresh; the skin finish is shiny, present and greasy.

Sensory nature of the formulae with 1% of emulsifiers: The application is slippery and fresh, the skin finish is comfortable but nonshiny, soft and nongreasy.

The HIP emulsions according to the invention (Ex42, Ex44, Ex46), characterized by an emulsifiers/squalane ratio by weight of less than 1/5, exhibit the nongreasy effect desired by the present invention. On the contrary, the HIP emulsions outside the invention of the comparative compositions Cp41, Cp43 or Cp45, which exhibit a ratio by weight of emulsifiers/squalane of greater than 1/5, exhibit a totally unacceptable greasy skin finish.

### Role of Different Systems Having a Preservative Effect for the Formula

Several ingredients were tested in order to evaluate their impact on the stability of the formulae. The use of salicylic acid, or of the following combinations, had no impact on the stability of the compositions according to the invention.

The following combinations were each tested as replacement for salicylic acid in the formulae of table 7:
Glutamic acid, N,N-diacetic acid, tetrasodium salt 0.2%
Glutamic acid, N,N-diacetic acid, tetrasodium salt 0.2%, salicylic acid 0.2%
Glutamic acid, N,N-diacetic acid, tetrasodium salt 0.2%, glyceryl caprylate 0.3%, vanillin 0.25%
   Conclusion: These reservative systems protect the formula without affecting the stability.

### Stability of the Compositions According to the Invention

The HIP emulsions according to the invention, in particular those of the examples (“Ex”) tested above, have proved to be stable over time, even at a temperature greater than ambient temperature (for example 45° C.): After 2 months of storage at all temperatures between 4° C. and 45° C., the compositions of the examples according to the invention (denoted Ex) exhibited no macroscopic change in color, odor or viscosity.

### Examples of Compositions According to the Invention

The content of aqueous phase is 70% and 85% respectively in the formulae of table 8 below.

TABLE 8

| INCI US | Ex 47 | Ex 48 |
|---|---|---|
| Squalane | 18 | |
| Undecane (and) Tridecane | | 11 |
| Polyglyceryl-4 Isostearate | 1 | 1 |
| Polyglyceryl-3 Diisostearate | 1 | 1 |
| Glycerol | 7 | 5 |
| Magnesium Sulfate | 0.7 | 0.7 |
| Propanediol | 5 | 5 |
| Salicylic Acid | 0.2 | 0.2 |
| Water | 66.9 | 75.9 |
| Tetrasodium Glutamate Diacetate | 0.2 | 0.2 |

The formulae are stable at 4° C., at ambient temperature (25° C.) and at 45° C., for at least 2 months.

### Comparison of Combinations of Emulsifiers According to the Invention and Outside the Invention

TABLE 9

| INCI US | Ex 48 | Cp 49 |
|---|---|---|
| Undecane (and) Tridecane | 11 | 11 |
| Polyglyceryl-4 Isostearate | 1 | 1 |
| Polyglyceryl-3 Diisostearate | 1 | |
| Inulin Lauryl Carbamate | | 1 |
| Glycerol | 5 | 5 |
| Magnesium Sulfate | 0.7 | 0.7 |
| Propanediol | 5 | 5 |
| Salicylic Acid | 0.2 | 0.2 |
| Water | 75.9 | 75.9 |
| Tetrasodium Glutamate Diacetate | 0.2 | 0.2 |

The formula of the example according to the invention Ex48 is stable at 4° C., at ambient temperature (25° C.) and at 45° C., for at least 2 months.

On the contrary, the formula of the comparative example outside the invention Cp49 is not stable; it shows a sedimentation in the cycles, at 2 months at ambient temperature and at 4° C., and a coalescence at 2 months at 45° C.

With otherwise identical W/O compositions, and including at least 70% of water, that, Cp49, using the emulsifiers polyglyceryl-4 diisostearate/polyhydroxystearate/sebacate and modified polysaccharide (outside the invention) is not stable, whereas that using, on the other hand, a combination of two polyglycerol ester emulsifiers according to the present invention is stable.

1. A composition in the form of a water-in-oil emulsion comprising:
a) a continuous oily phase containing:
at least two emulsifiers chosen from esters of C₈-C₃₀ fatty acid and of polyglycerol, and ps at least one nonsilicone oil with a density relative to water at 4° C. of less than 0.9; and
b) an aqueous phase dispersed in said oily phase and representing a content of at least 70% by weight, with respect to the total weight of the composition representing 100%;
characterized in that the ratio by weight of said emulsifiers to said at least one oil is less than 1/5.

2. The composition as claimed in claim 1, in which the ratio by weight of said emulsifiers to said at least one oil is between 1/25 and 1/5.

3. The composition as claimed in claim 1, in which the ratio by weight of the aqueous phase to the oily phase is within the range from 70/30 to 95/5.

4. The composition as claimed in claim 1, in which the aqueous phase represents from 74% to 95%, by weight, with respect to the total weight of the composition representing 100%.

5. The composition as claimed in claim 1, in which said at least two emulsifiers represent a total content by weight of active material of less than 5%, by weight, with respect to the total weight of the composition.

6. The composition as claimed in claim 1, in which said fatty acid of said emulsifiers comprises a C₁₆-C₂₀ hydrocarbon chain.

7. The composition as claimed in claim 1, in which said esters of fatty acid and of polyglycerol each independently contain from 2 to 10 glycerol units.

8. The composition as claimed in claim 1, in which said at least two esters of fatty acid and of polyglycerol are independently chosen from: esters of isostearic acid and of polyglycerol.

9. The composition as claimed in claim 1, characterized in that the ratio by weight between said emulsifiers is within the range from 1:2 to 2:1.

10. The composition as claimed in claim 1, in which the two emulsifiers are Polyglyceryl-4 Isostearate and Polyglyceryl-3 Diisostearate.

11. The composition as claimed in claim 1, in which the two emulsifiers are Polyglyceryl-3 Diisostearate and Polyglyceryl-4 Diisostearate/Polyhydroxystearate/Sebacate.

12. The composition as claimed in claim 1, in which the two emulsifiers are Polyglyceryl-4 Isostearate and Polyglyceryl-4 Diisostearate/Polyhydroxystearate/Sebacate.

13. The composition as claimed in claim 1, characterized in that said at least one oil is chosen from C₈-C₃₀ alkanes and/or dialkyl carbonates of respective C₈-C₃₀ alkyl chains and/or fatty esters of formula R₁COOR₂ where R₁ represents the residue of a fatty acid comprising from 8 to 30 carbon atoms and R₂ represents a linear or branched hydrocarbon chain containing from 6 to 30 carbon atoms.

14. The composition as claimed in claim 1, characterized in that said at least one oil is chosen from squalane, linear or branched C₈-C₁₂ alkanes and their mixtures, C₁₅-C₂₈ alkanes and their mixtures, dicaprylyl carbonate, C₁₆-C₂₀ fatty acid esters and the mixtures of these oils.

15. The composition as claimed in claim 1, characterized in that it contains less than 1% of polymers and/or of silicone oil, and/or of gelling agent or thickener, and/or of fatty substances of synthetic origin, and/or of any other fatty substance other than an oil with a relative density of less than 0.9 and chosen from said alkanes and/or said dialkyl carbonates and/or said fatty esters.

16. The composition as claimed in claim 1, characterized in that the aqueous phase additionally comprises from 0.5% to 20% of at least one polyol, the percentages being by weight, with respect to the total weight of the composition representing 100%.

17. The composition as claimed in claim 1, characterized in that it additionally comprises at least one cosmetic adjuvant chosen from cosmetic or dermatological active principles, preservatives, antioxidants, fragrances, fillers, pigments, UV screening agents, odor absorbers and coloring materials.

18. The composition as claimed in claim 1, characterized in that it additionally comprises from 0.2% to 2% the percentages being by weight, with respect to the total weight of the composition representing 100%.

19. The composition as claimed in claim 1, characterized in that it additionally comprises from 0.1% to 1% of at least one compound chosen from: salicylic acid, glutamic acid, N,N-diacetic acid, tetrasodium salt, pentylene glycol, glyceryl caprylate, vanillin and their mixtures, the percentages being by weight, with respect to the total weight of the composition representing 100%.

20. The composition as claimed in claim 1, characterized in that it additionally comprises at least one active principle chosen from moisturizing agents; agents for combating free radicals; keratolytic and desquamating agents; vitamins; antielastase and anticollagenase agents; trace elements; algal or plankton extracts; enzymes and coenzymes; flavonoids and isoflavonoids; ceramides; antiglycation agents; NO-synthase inhibitors; agents which stimulate the synthesis of dermal or epidermal macromolecules and/or which prevent their degradation; agents which stimulate the proliferation of fibroblasts or keratinocytes and/or the differentiation of keratinocytes; tightening agents; agents for combating pollution and/or free radicals; and muscle relaxants or dermo-decontracting agents; and their mixtures.

21. An oily emulsifying composition, characterized in that it has a composition in accordance with the oily phase a) of claim 1.

22. The use of an oily emulsifying composition as claimed in claim 21 in the manufacture of a water-in-oil HIPE emulsion with improved stability and/or having a reduced greasy effect, and with a content of aqueous internal phase of at least 70% by with respect to the total weight of the emulsion.

23. A process for the manufacture of a water-in-oil emulsion as claimed in claim 1, in which said homogeneous aqueous phase is mixed with said oily phase at ambient temperature, within the range from 15 to 25° C.

24. A kit comprising:
an oily emulsifying composition with a composition in accordance with the oily phase (a) of claim 1, and
an aqueous composition (b) as defined in claim 1,
in which said respectively aqueous composition b) and said respectively oily composition (a) are packaged separately and intended to be mixed at the time of use in order to form a cosmetic product.

25. A nontherapeutic method for caring for, making up or removing makeup from the skin, hair and/or lips, comprising the application, to the skin, hair and/or lips, of an emulsion according to claim 1.
